(19) United States
(12) Patent Application Publication
(10) Pub. No.: US 2021/0228105 A1
(43) Pub. Date: Jul. 29, 2021

(54) METHODS FOR DETERMINING STOMACH FULLNESS AND RELATED METHODS OF MEDICAL SAFETY

(71) Applicant: Amir Marashi, Charlotte, NC (US)

(72) Inventor: Amir Marashi, Charlotte, NC (US)

(21) Appl. No.: 17/050,108

(22) PCT Filed: Apr. 24, 2019
(86) PCT No.: PCT/US2019/028827

§ 371 Date: Oct. 23, 2020

Related U.S. Application Data
(60) Provisional application No. 62/663,519, filed on Apr. 27, 2018.

(51) Int. Cl. A61B 5/083; A61B 5/097; A61B 5/00

(52) U.S. Cl. CPC A61B 5/083; A61B 5/7275; A61B 5/097

(57) ABSTRACT

A method of determining a fullness level of an organism's stomach comprises obtaining a specimen from the organism, measuring an amount of one or more analytes in the specimen, and determining the fullness level of the organism's stomach based on the measurement of the amount of the one or more analytes.

### CROSS REFERENCE TO RELATED APPLICATIONS

This application claims priority pursuant to 35 U.S.C. § 119 to U.S. Provisional Patent Application No. 62/663,519, filed on Apr. 27, 2018, which is hereby incorporate by reference in its entirety.

### FIELD

The invention is generally related to methods for determining a fullness level of a stomach, and more specifically to anesthesia safety.

### BACKGROUND

Certain medical procedures require a patient have an empty stomach prior to commencing the medical procedure. In such instances, patients are instructed to not eat or drink anything by mouth during the preceding 8 to 12 hours prior to the scheduled medical procedure. Not following this instruction can result in delay of the medical procedure, rescheduling the medical procedure, an unsuccessful medical procedure, or life threatening complications during the medical procedure.

Exemplary procedures requiring patients to following these instructions include any procedures wherein the patient receives general anesthesia. While general anesthesia poses a multitude of life threatening risks and dangers to patients, some may be preventable. An example of a preventable complication is acid aspiration syndrome, wherein contents originating in the patient's stomach ultimately occlude the patient's airway, thereby preventing respiratory function, causing asphyxiation, and resulting in general hypoxia. A patient's risk of developing acid aspiration syndrome is directly related to the fullness of the patient's stomach. Prior to receiving general anesthesia, a patient is often asked whether or not they have consumed any food or drink in the last 8 to 12 hours. In some instances, a patient may forget to report something they consumed, or perhaps in fear of consequences including canceling or delaying their schedule procedure, may intentionally lie or fail to report having eaten or drank during the time frame in question. In other instances, the patient may be incapable of communicating that they had eaten or drank in the preceding hours of a scheduled or even an unanticipated medical procedure. In any situation wherein a patient receives general anesthesia and an inaccurate or erroneous report of whether or not the patient has eaten or drank anything in the previous 8 to 12 hours is provided to the medical professional, the patient's life may be at undue risk.

### SUMMARY

In one aspect, methods for determining the fullness level of an organism's stomach are described herein, which in some embodiments can offer one or more advantages over current methods. The methods described herein can be utilized in the practice of human and/or veterinary medicine. For example, methods described herein can, in some embodiments, provide objective information without relying on the patient's signs and symptoms or information from a subjective questionnaire. In another embodiment, methods described herein can be used when the patient is unable to communicate the information needed to ensure their safety during the medical procedure. In some instances, methods described herein can provide objective information which can be utilized in combination with the patient's medical history to provide a more complete medical profile. Methods described herein are, in some instances, performed rapidly and non-invasively. Additionally, in some embodiments, methods described herein are conducive to practicing personalized medicine, such that a personalized profile of measured analytes in combination with a patient's medical history can provide medically relevant information about the patient that is objective and specific to that patient.

In some embodiments, a method of determining a fullness level of an organism's stomach described herein comprises obtaining a specimen from the organism, measuring an amount of one or more analytes in the specimen, and determining the fullness level of the organism's stomach based on the measurement of the amount of the one or more analytes. In some embodiments, obtaining a specimen includes obtaining an oral specimen. For example, in some instances, an oral specimen is a saliva sample, a buccal swab, or a pulmonary expiration sample. A pulmonary expiration sample can, in some embodiments, be provided directly into an analyzing device. In other instances, obtaining the specimen includes obtaining a blood sample.

A method described herein, in some embodiments, comprises measuring one or more analytes in the specimen that are an inorganic, an organic, or a combination of both. In some embodiments, the one or more analytes are produced by a digestive or metabolic process within the organism. For example, the one or more analytes can be produced by the organism's microbiome. Measuring one or more analytes, in other instances, further comprises determining that one or more analytes is absent from the specimen or present in the specimen below a detectable level. In some embodiments, a plurality of amounts of a plurality of analytes is measured.

In some embodiments, a method described herein further comprises correlating the measurement of the amount of the one or more analytes to a reference profile of analyte amounts. For example, in some instances, the fullness level is a qualitative measurement of gastric volume. In other instances, the fullness level is a binary fullness level corresponding to whether or not the organism's stomach contains contents above a minimal threshold level.

In another aspect, methods described herein, allow medical professionals to prevent and/or identify procedural risks and complications, including life threatening risks such as acid aspiration syndrome. In some embodiments, a method described herein of preventing acid aspiration syndrome in a patient comprises obtaining a specimen from the patient, measuring an amount of one or more analytes in the specimen, determining a fullness level of the patient's stomach based on the measurements of the amount of the one or more analytes, and determining the patient's level of risk to receive general anesthesia based on the fullness level of the patient's stomach, wherein the specimen is collected about 12 hours or less prior to receiving general anesthesia.

In some embodiments, obtaining a specimen from the patient comprises obtaining an oral specimen. For example, in some instances, an oral specimen is a saliva sample, a buccal swab, or a pulmonary expiration sample. A pulmonary expiration sample can, in some cases, be provided directly into an analyzing device. In other instances, obtaining the specimen includes obtaining a blood sample from the patient.

A method described herein, in some embodiments, comprises measuring one or more analytes in the specimen that are inorganic, organic, or a combination of both. In other embodiments, the one or more analytes are produced by a digestive or metabolic process within the organism. For example, the one or more analytes can be produced by the patient's microbiome. In another example, the one or more analytes are related to the patient's metabolic state. Measuring one or more analytes, in some instances, further comprises determining that one or more analytes is absent from the specimen or present in the specimen below a detectable level. In some embodiments, a plurality of amounts of a plurality of analytes is measured.

In some embodiments, a method described herein further comprises correlating the measurement of the amount of the one or more analytes to a reference profile of analyte amounts. For example, in some instances, the fullness level is a qualitative measurement of gastric volume. In other instances, the fullness level is a binary fullness level corresponding to whether or not the patient's stomach contains contents above a minimal threshold level.

These and other embodiments are described in greater detail in the detailed description which follows.

### DETAILED DESCRIPTION

Embodiments described herein can be understood more readily by reference to the following detailed description and examples. Elements and methods described herein, however, are not limited to the specific embodiments presented in the detailed description and examples. It should be recognized that these embodiments are merely illustrative of the principles of the present disclosure. Numerous modifications and adaptations will be readily apparent to those of skill in the art without departing from the spirit and scope of the disclosure.

In addition, an amount described herein can be an absolute amount or a relative amount. An amount can be expressed on an arbitrary scale according to a range. For example, an amount can be an absolute value, as measured in standard units of measurement for an analyte. In another example, an amount can be a percentage of a normalized or controlled reference amount. In yet another example, an amount can be quantified as it relates to a known range, such that the amount is an assigned value according to its position on an arbitrary scale that is synonymous to a known range. The quantification of an amount can vary based on the measured analyte.

### I. Methods of Determining a Fullness Level of a Stomach

In one aspect, methods of determining a fullness level of an organism's stomach are described herein. In some embodiments, a method comprises obtaining a specimen from an organism, measuring an amount of one or more analytes in the specimen, and determining the fullness level of the organism's stomach based on the measurement of the amount of the one or more analytes. In some cases, a method described herein further comprises determining that one or more analytes is absent from the specimen or present in the specimen below a detectable level. Additionally, a method described herein can further comprise correlating the measurement of the amount of the one or more analytes to a reference profile of analyte amounts.

In some embodiments, an organism comprises an animal. For example, an organism can be any animal having a stomach, such a mammal. In another embodiment, an organism can be a human. The organism can be of any size or mass, notwithstanding that the organism has a stomach. Obtaining a specimen from an organism, in some instances, is performed in the context of veterinary study and/or veterinary medicine. In some cases, an animal can be a domesticated animal or a wild animal. For example, an animal, in some instances, is a pet or a wild-caught animal. In other instances, obtaining a specimen from an organism is performed in the context of human study and/or human medicine. In some embodiments, a human or other animal is ill, and in other embodiments, a human or other animal is not ill, or is considered otherwise healthy.

In some embodiments, a specimen comprises a biological specimen, or sample, or any part collected from or extracted from the organism. For example, a specimen can be an oral specimen, a sputum specimen, a blood specimen, a urine specimen, a tissue specimen, a fecal specimen, or any other similar specimen type. An oral specimen can comprise any specimen taken, captured, or isolated from an oral cavity. For example, in some cases, an oral specimen is a saliva sample, a buccal swab, or a pulmonary expiration sample. Obtaining a specimen, in some embodiments, comprises using non-invasive techniques. For example, obtaining a specimen can comprise obtaining the specimen from a bodily function. In some embodiments, a specimen is obtained by voluntary or involuntary bodily functions, such as micturition, bowel movement, cough, respiration, expiration, sweat, or other bodily function. Additionally, in some instances, a pulmonary expiration occurring from respiration can be provided directly into an analyzing device, wherein the analyzing device facilitates measuring an amount of the one or more analytes in the specimen. For example, a person can exhale directly into the analyzing device. Obtaining a specimen, in other embodiments, comprises using invasive techniques. For example, obtaining a specimen can comprise collecting the specimen using a needle. In some embodiments, the needle is inserted into a blood vessel to obtain a blood specimen. In other embodiments, the needle is inserted in a biological tissue to obtain a tissue specimen.

Methods described herein, in some embodiments, comprise measuring an amount of one or more analytes in the specimen. In some cases, the one or more analytes are an inorganic, an organic, or a combination of both. In some embodiments, the one or more analytes are from food or drink consumed by the organism. In other instances, the one or more analytes are produced by a digestive or metabolic process within the organism. For example, an organism comprising a digestive tract can exhibit metabolic activity or a biological response following consumption of food or drink. The metabolic activity or biological response can result in forming an amount of one or more analytes. In some instances, metabolic activity or biological response can occur in the oral cavity, stomach, intestine, or other sections of the digestive tract. In other instances, biological responses can occur in the blood or a metabolically active tissue, wherein the metabolically active tissue is triggered by a digestive process.

In another embodiment, the one or more analytes are produced in a state of fasting. For example, in some instances a metabolic process can occur as a biological response to a depletion of energy, such as gluconeogenesis or ketosis. Therefore, the one or more analytes can, in some instances, be produced from the organism only. In another embodiment, the one or more analytes are produced by the organism's microbiome. For example, an organism having a digestive tract comprising a microbiome can produce a specimen comprising an analyte formed from metabolically active microbiota. An analyte formed from microbiota can, in some cases, only be formed from the microbiota. For example, the microbiota can possess enzymatic processes that are not in common or not shared with the organism. The one or more analytes, in other embodiments, can be formed from the metabolic activity of the organism and the microbiota. For example, in some instances a substrate is first enzymatically modified by the organism to generate a first product, and the first product is subsequently enzymatically modified by the microbiota to form a second product, or vice versa, wherein the second product is the analyte. Non-limiting examples of analytes are described below in Table 1, although any analyte not inconsistent with the goal of this disclosure are also contemplated. Furthermore, it should be understood by one of ordinary skill in the art that detection of any analyte not inconsistent with the scope of the invention can be used.

TABLE 1

| Hormones | Enzymes | Other |
|---|---|---|
| Steroid | Amylase | Glucose |
| Eicosanoid | Peptidase | Fructose |
| Leukotriene | Catalase | Amino acid |
| Prostaglandin | Trypsin | Peptide |
| Thyroid simulating | Trypsinogen | Fatty acid |
| hormone/thyrotropin | | |
| Thyroxine | Lipase | Lipoprotein |
| Thyrotropin releasing | Cellulase | Ketones |
| hormone | | |
| Triiodothyronine | Protease | H+ ion |
| Vasopressin | Pectinase | Sugar molecule |
| Catecholamine | Lactase | |
| Renin | Cathepsin | |
| Angiotensin | Bromelain | |
| Leptin | Papain | |
| Insulin | Glucoamylase | |
| Ghrelin | Aminopeptidase | |
| Neuropeptide Y | Carboxypeptidase | |
| Gastrin | Chymotrypsin | |
| Cholecystokinin | Dipeptidase | |
| Gastric Inhibitor Peptide | Elastase | |
| Secretin | Enterokinase | |
| Motilin | Gastric lipase | |
| | Lingual lipase | |
| | Maltase | |
| | Pepsin | |
| | Sucrase | |
| | Alpha-Dextrinase | |

In another embodiment, measuring an amount of one or more analytes in the specimen further comprises determining that one or more analytes is absent from the specimen or present in the specimen below a detectable level. A detectable level, in some cases, can be dependent on the method of detection. For example, a method of detection can include enzymatic assay, immunoassay, photovoltaic assay, semiconductor gas sensing, electrochemical assay, nanopore assay, chemical reaction assays, chemical separation methods (e.g., high pressure liquid chromatography, mass spectrometry, nuclear magnetic resonance, and others), or other analytical assay. Any method of detection not inconsistent with the scope of the invention can be used to measure the one or more analytes.

In another embodiment, methods described herein comprise measuring an amount of one or more analytes in the specimen, wherein a plurality of amounts of a plurality of analytes is measured. For example, methods described herein can further comprise forming a profile. A profile, in some cases, comprises a plurality of measurements corresponding to a plurality analytes. For example, a profile, in some embodiments, comprises an amount of a first analyte, an amount of a second analyte, an amount of a third analyte, an amount of a fourth analyte, an amount of a fifth analyte, and/or n amounts of up to n analytes. In some embodiments, a profile comprises n amounts of n analytes, wherein each of n amounts is a measurement of each of n analytes. The one or more analytes need not be measured in the same unit of measurement. For example, each analyte can be measured in its known standardized unit of measure. In some embodiments, the one or more analytes are related to the metabolic state of the organism. In some embodiments, a profile comprising n amounts of n analytes can be normalized or modified according to the organism. For example, in some cases, a profile can be modified according to known a health condition of the organism. In another example, a profile can be modified according to a sex, a size, a mass, and/or an age of the organism. In yet another example, a profile can be normalized, modified, or compared to a baseline profile, wherein a baseline profile comprises measurements of the same analytes measured at a previous point in time. The previous point in time, in some instances, represents a time when the organism was in a different or similar metabolic state.

In other embodiments, methods described herein comprise determining the fullness level of the organism's stomach base on the measurements of the one or more analytes. For example, determining the fullness level can comprise correlating the measurement of the amount of the one or more analytes to one or more reference measurements of analyte amounts. In some instances, the one or more reference measurements can form a reference profile. A reference profile, in some embodiments, can be related to the species of the organism. In other embodiments, a reference profile can be normalized according to the health and/or medical history of the organism. For example, in some cases, a reference profile can be modified according to known medical conditions. In another example, a reference profile can be modified according to sex, size, mass, and/or age. In other embodiments, a reference profile can be a baseline profile. A baseline profile, in some embodiments, comprises measurements of the same analytes that were measured at a previous point in time. The previous point in time, in some instances, represents a time when the organism was in a different or similar metabolic state.

The fullness level, in some embodiments, is a qualitative measurement of gastric volume. For example, the fullness level, in some embodiments, can correspond to the volume of contents relative to the total volumetric capacity of the stomach. In another embodiment, the fullness level is a binary fullness level corresponding to whether or not the organism's stomach contains contents above a minimal threshold level. A minimal threshold level, in some embodiments, can be any threshold level below full capacity. Additionally, a minimal threshold level, in some embodiments, can depend on the type of organism and/or the characteristics of the organism. For example, a minimal threshold can vary by species. A minimal threshold, in some embodiments, can vary by sex, size, age, and/or weight of the organism.

### II. Methods of Preventing Acid Aspiration Syndrome

In another aspect, methods of preventing acid aspiration syndrome in a patient are described herein. In some embodiments, a method comprises obtaining a specimen from the patient, measuring an amount of one or more analytes in the specimen, determining a fullness level of the patient's stomach based on the measurements of the amount of the one or more analytes, and determining the patient's level of risk to receive general anesthesia based on the fullness level of the patient's stomach, wherein the specimen is collected about 12 hours or less prior to receiving general anesthesia. In another embodiment, methods described herein comprise methods for objectively determining whether or not a patient has orally consumed any food or drink in the last 12 hours or less. In some embodiments, methods described herein comprise methods of preventing Mendelson's syndrome, chemical pneumonitis, or aspiration pneumonitis. Methods described herein can be used to prevent a patient from general hypoxia related to the aspiration of gastric contents or other fluids, such as blood, bile, water, acid, or other fluid. Additionally, for example, methods described herein can be used to identify a fasting state of a patient prior to receiving general anesthesia to prevent procedural complications.

In some embodiments, a patient can be any patient under the care of a medical professional, including, in some instances, a professional of veterinary medicine (“D.V.M.”) or a medical doctor (“M.D.”). In some embodiments, the patient can be in preparation for a surgical procedure. A surgical procedure, in certain instances, comprises any medical procedure requiring general anesthesia. In other instances, a surgical procedure comprises a medical procedure for which the patient voluntarily elected to receive general anesthesia, even though general anesthesia may not necessarily be required to perform the procedure.

In some embodiments, a specimen comprises a biological specimen, or sample, or any part collected from or extracted from the patient. For example, a specimen can be an oral specimen, a sputum specimen, a blood specimen, a urine specimen, a tissue specimen, a fecal specimen, or any other specimen type. An oral specimen, in some embodiments, can comprise any specimen taken, captured, or isolated from an oral cavity. For example, in some cases, an oral specimen is a saliva sample, a buccal swab, or a pulmonary expiration sample. Obtaining a specimen, in some embodiments, comprises using non-invasive techniques. For example, obtaining a specimen can comprise obtaining the specimen from a bodily function. In some embodiments, a specimen is obtained by voluntary or involuntary bodily functions, such as micturition, bowel movement, cough, respiration, expiration, sweat, or other bodily function. Additionally, in some instances, a pulmonary expiration occurring from respiration can be provided directly into an analyzing device. For example, a person can exhale directly into the analyzing device. Obtaining a specimen, in other embodiments, comprises using invasive techniques. For example, obtaining a specimen can comprise collecting the specimen using a needle. In some embodiments, the needle is inserted into a blood vessel to obtain a blood specimen. Therefore, in some embodiments, the specimen is a blood sample. In other embodiments, the needle is inserted in a tissue to obtain a tissue specimen. Therefore, in other embodiments, the specimen is a tissue sample.

In other embodiments, measuring an amount of one or more analytes in the specimen can comprise an amount of an inorganic analyte, an organic analyte, or a combination of both. Non-limiting examples of analytes are described in Table 1 above, although any analyte not inconsistent with the goals of this disclosure are also contemplated. In some embodiments, measuring one or more analytes includes determining the absence or undetectable levels of the one or more analytes. In other embodiments, the amount of one or more analytes can comprise analyte from food or drink recently consumed by the patient. The term “recently consumed” can be within the last 24 hours, 12 hours, 8 hours, or 4 hours prior to obtaining the specimen. An amount of one or more analytes, in some embodiments, can comprise an amount of one or more fats, carbohydrates, proteins, minerals, vitamins, or combination thereof. In another embodiment, the one or more analytes are produced from a digestive or metabolic process of the patient. The digestive or metabolic process, in some embodiments, is a biological response to previously consumed food or drink. In other embodiments, the digestive or metabolic process is a biological response to the absence of energy or a depleted state of energy, such as gluconeogenesis or ketosis. For example, a metabolic process, in some instances, can be a metabolic state of fasting. Thus, the one or more analytes, in some embodiments, can be related to the patient's metabolic state. In some embodiments, the digestive or metabolic process can occur in any enzymatically active environment of the digestive tract. For example, the process can occur in the oral cavity, the stomach, or the gut. Therefore, an amount of one or more analytes can comprise an amount of one or more enzymes. The one or more enzymes, in some embodiments, are enzymes involved in the digestive process. In other embodiments, the digestive or metabolic process can occur outside the digestive tract. For example, a digestive or metabolic process can be any endocrine or otherwise actively secretory process, such as an activity of the endocrine or exocrine system. Therefore, an amount of one or more analytes can comprise an amount of one or more hormones. The one or more hormones, in some embodiments, can be related to the patient's metabolic state. In other instances, the one or more analytes are produced by the patient's microbiome. In some instances, the one or more analytes can be a product from enzymatic activity found only within the patient's microbiome. For example, in some instances a substrate is first enzymatically modified by the patient to generate a first product and the first product is subsequently enzymatically modified by the microbiota to form a second product, or vice versa, wherein the second product is the analyte. In still other instances, the one or more analytes can be a product from enzymatic activity of both the patient's body and the patient's microbiome. In another instance, an amount of one or more analytes can be a product of enzymatic activity of only the patient's body.

In some embodiments, a plurality of amounts of a plurality of analytes is measured. The analytes can be measured using one or more of the methods previously described herein. The measuring of the plurality of amounts of the plurality of analytes can comprise forming a profile. In some embodiments, a profile comprises an amount of a first analyte, an amount of a second analyte, an amount of a third analyte, an amount of a fourth analyte, an amount of a fifth analyte, and/or n amounts of up to n analytes. In some embodiments, a profile comprises n amounts of n analytes, wherein each of n amounts is a measurement of each of n analytes. The one or more analytes can be measured in the same unit of measurement or can be measured in different units of measurement. For example, each analyte can be measured in its known standardized unit of measure. A profile, in some cases, comprises a plurality of measurements corresponding to a plurality analytes. In some embodiments, a profile can be normalized or modified according to the health and/or medical history of the patient. For example, in some cases, a profile can be modified according to a known health condition of the patient. In another example, a profile can be modified according to a sex, height, weight, and/or age of the patient. In yet another example, a profile can be normalized, modified, or compared to a baseline profile, wherein a baseline profile comprises measurements of the same analytes that were measured at a previous point in time. The previous point in time, in some instances, represents a time when the patient was in a different or similar metabolic state.

In other embodiments, methods described herein comprise determining a fullness level of the patient's stomach base on the measurements of the one or more analytes. For example, determining the fullness level can comprise correlating the measurements of the amount of the one or more analytes to one or more reference measurements of the one or more analyte amounts. In some instances, the one or more reference measurements can comprise a reference profile. A reference profile, in some embodiments, can comprise a baseline profile of the same patient. A baseline profile comprises measurements of the same one or more analytes measured at a previous point in time. The previous point in time, in some instances, represents a time when the patient was in a different or similar metabolic state. A reference profile, in some embodiments, can be a profile according to the health and/or medical history of the patient. For example, in some cases, a reference profile can be a profile modified according to known medical conditions. Examples of known medical conditions can include medical conditions related to metabolism. For example, a reference profile can be a profile modified according to metabolic syndrome, diabetes, obesity, bulimia, anorexia, or other metabolic disease or food-related disorder. In another example, a reference profile can be a profile modified according to a sex, height, weight, and/or age. The fullness level, in some embodiments, is a qualitative measurement of gastric volume. For example, the fullness level, in some embodiments, can correspond to the volume of contents relative to a total volumetric capacity of the patient's stomach. A total volumetric capacity, in some embodiments, can depend on the patient's sex, height, weight, age, and/or health condition. In another embodiment, the fullness level is a binary fullness level corresponding to whether or not the patient's stomach contains residual gastric contents above a minimal threshold level. A minimal threshold level, in some embodiments, can depend on the patient's sex, height, weight, and/or age. Additionally, a minimal threshold level, in some embodiments, can be any threshold level below full capacity. For example, a minimal threshold, in some embodiments, can be at least 1.5 ml per kg of patient weight. In other embodiments, a minimal threshold is at least 25 mL of residual gastric volume. In some instances, the minimal threshold can further depend on the pH of the stomach. For example, a minimal threshold can be at least 25 mL with a pH of less than 2.5.

In another embodiment, methods described herein comprise determining the patient's level of risk to receive general anesthesia based on the fullness level of the patient's stomach. In some embodiments, the patient's level of risk directly corresponds with the fullness level. For example, a high fullness level can correspond to a high level of risk. In another example, a moderate fullness level can correspond to a moderate level of risk. In yet another example, a low fullness level can correspond to a low level of risk. In another embodiment, the patient's level of risk can be binary and correspond to a binary fullness level corresponding to whether or not the patient's stomach contains residual gastric contents above a minimal threshold level. For example, a patient's level of risk can be high if the level of fullness is at or above the minimal threshold level. Alternatively, the patient's level of risk can be low if the level of fullness is at or below the minimal threshold level. In some embodiments, determining a patient's level of risk further comprises determining whether or not to administer general anesthesia to the patient. For example, a moderate to high level of risk can indicate it is not safe to administer anesthesia, whereas a low level of risk can indicate it is relatively safe to administer anesthesia.

1. A method of determining a fullness level of an organism's stomach, the method comprising:
obtaining a specimen from the organism;
measuring an amount of one or more analytes in the specimen; and
determining the fullness level of the organism's stomach based on the measurement of the amount of the one or more analytes wherein the specimen is a pulmonary expiration sample.

2-3. (canceled)

4. The method of claim 1, wherein the pulmonary expiration sample is provided directly into an analyzing device.

5. (canceled)

6. The method of claim 1, wherein the one or more analytes are an inorganic, an organic, or a combination of both.

7. The method of claim 1, wherein the one or more analytes are digestive or metabolic products.

8. The method of claim 7, wherein the one or more analytes are a microbiome product.

9. The method of claim 1, wherein measuring an amount of the one or more analytes comprises determining that the one or more analytes is absent from the specimen or present in the specimen below a detectable level.

10. The method of claim 1, wherein a plurality of amounts of a plurality of analytes is measured.

11. The method of claim 1, wherein determining the fullness level comprises correlating the measurement of the amount of the one or more analytes to a reference profile of analyte amounts.

12. The method of claim 1, wherein the fullness level is a qualitative measurement of gastric volume.

13. The method of claim 1, wherein the fullness level is a binary fullness level corresponding to whether or not the organism's stomach contains contents above a minimal threshold level.

14. A method of preventing acid aspiration syndrome in a patient, the method comprising: obtaining a specimen from the patient;
measuring an amount of one or more analytes in the specimen;
determining a fullness level of the patient's stomach based on the measurements of the amount of the one or more analytes; and
determining a patient risk level to receive general anesthesia based on the fullness level of the patient's stomach,
wherein the specimen is collected about 12 hours or less prior to receiving general anesthesia, and
wherein the specimen is a pulmonary expiration sample.

15-16. (canceled)

17. The method of claim 14, wherein the pulmonary expiration sample is provided directly into an analyzing device.

18. (canceled)

19. The method of claim 14, wherein the one or more analytes are an inorganic, an organic, or a combination of both.

20. The method of claim 14, wherein the one or more analytes are a digestive or metabolic product.

21. The method of claim 20, wherein the one or more analytes are a microbiome product.

22. The method of claim 20, wherein the one or more analytes are related to the patient's metabolic state.

23. The method of claim 14, wherein measuring one or more analytes includes determining the absence or undetectable levels of the one or more analytes.

24. The method of claim 14, wherein a plurality of amounts of a plurality of analytes is measured.

25. The method of claim 14, wherein determining the fullness level comprises correlating the measurement of the amount of the one or more analytes to a reference profile of analyte amounts.

26. The method of claim 14, wherein the fullness level is a qualitative measurement of gastric volume.

27. The method of claim 14, wherein the fullness level is a binary fullness level corresponding to whether or not the patient's stomach contains contents above a minimal threshold level.
